(19) United States
(12) Patent Application Publication
(10) Pub. No.: US 2025/0352700 A1
(43) Pub. Date: Nov. 20, 2025

(54) INJECTABLE DOSIMETER COMPOSITIONS AND METHODS OF USING SAME

(71) Applicants: Rowan University, Glassboro, NJ (US); The Cooper Health System, Camden, NJ (US)

(72) Inventors: Sebastian L. Vega, Blackwood, NJ (US); Kirstene Gultian, Glassboro, NJ (US); Leonard Kim, Pennington, NJ (US); Irina Malajovich, Swarthmore, PA (US); Geoffrey S. Ibbott, Jericho, VT (US)

(73) Assignees: Rowan University, Glassboro, NJ (US); The Cooper Health System, Camden, NJ (US)

(21) Appl. No.: 18/871,341

(22) PCT Filed: Jun. 2, 2023
(86) PCT No.: PCT/US2023/024285

§ 371 Date: Dec. 3, 2024

Related U.S. Application Data
(60) Provisional application No. 63/348,920, filed on Jun. 3, 2022.

(51) Int. Cl. A61L 27/52; C08B 37/08; C08J 3/075; G01T 1/04

(52) U.S. Cl. CPC A61L 27/52; C08B 37/0072; C08J 3/075; C08J 2305/00; G01T 1/04

(57) ABSTRACT

Described herein is a biocompatible injectable dosimeter. The injectable dosimeter comprises a polymer gel and a radiation dosimeter material distributed in the polymer gel. The dosimeter material in the injectable dosimeter, when radiated, generates a dosimetric signal, which can be used to evaluate the delivery of the radiation. Also described are methods of using the injectable dosimeter, such as to report radiation in real-time and to protect tissues adjacent to the disease sites being irradiated.

### CROSS-REFERENCE TO RELATED APPLICATIONS

The present application claims priority under 35 U.S.C. § 119 (e) to U.S. Provisional Patent Application No. 63/348,920, filed Jun. 3, 2022, which is incorporated herein by reference in its entirety.

### BACKGROUND

Radiotherapy is an effective treatment for many diseases, such as but not limited to various types of cancers. The effectiveness of radiotherapy depends on the delivery of radiation to the exact disease site and at precise dosage necessary to treat or ameliorate the disease. Importantly, radiotherapy can produce considerable damage to healthy tissue that is located in the proximity of disease sites. Therefore, there is a need for dosimetric compositions that can be used to evaluate delivery of radiation in a radiotherapy treatment. The present invention addresses this need.

### SUMMARY

In some aspects, the present invention is directed to the following non-limiting embodiments:

In some embodiments, the present invention is directed to an injectable dosimeter composition.

In some embodiments, the injectable dosimeter composition comprises a polymer gel.

In some embodiments, the injectable dosimeter composition further comprises a radiation dosimeter material distributed in at least a portion of the polymer gel.

In some embodiments, the radiation dosimeter material generates a dosimetric signal in response to a radiation applied to the polymer gel.

In some embodiments, the injectable dosimeter composition is biocompatible.

In some embodiments, the polymer gel comprises a crosslinked polymer gel.

In some embodiments, the polymer gel is crosslinked via a small molecule crosslinker, a reactive functional group attached to the polymer chain, a photo cross-linking group attached to the polymer chain, or an enzyme-catalyzed cross-linking reaction.

In some embodiments, the polymer gel comprises a hydrogel.

In some embodiments, the hydrogel comprises an alginate hydrogel, a carboxymethylcellulose hydrogel, a collagen hydrogel, a hyaluronic acid hydrogel, a polyethylene glycol (PEG) hydrogel, a poly (2-hydroxyethyl methacrylate) (pHEMA) hydrogel, a poly (2-hydroxypropyl methacrylate) hydrogel, a polymethylmethacrylate (PMMA) hydrogel, a poly-lactic acid (PLA) hydrogel, a polyacrylamide hydrogel, or combinations thereof.

In some embodiments, the polymer gel comprises a crosslinked hyaluronic acid polymer.

In some embodiments, the polymer gel comprises a hyaluronic acid polymer modified with a norbornene group and a hyaluronic acid polymer modified with a tetrazine group.

In some embodiments, the polymer gel is partially polymerized.

In some embodiments, the polymer gel is fully polymerized.

In some embodiments, injectable dosimeter composition has been injected into the body of a subject.

In some embodiments, the dosimetric signal generated in response to the radiation is substantially proportional to the radiation dose.

In some embodiments, the dosimetric signal generated in response to the radiation is detectable by magnetic resonance imaging (MRI) or any optical imaging method.

In some embodiments, the radiation comprises at least one selected from the group consisting of x-ray, gamma ray, electron beam, and proton beam.

In some embodiments, the radiation dosimeter material comprises a transition metal element.

In some embodiments, the radiation dosimeter material comprises Fe and/or Gd.

In some embodiments, the radiation dosimeter material comprises Fe (II), and wherein the radiation converts the Fe (II) to Fe (III).

In some embodiments, the radiation dosimeter composition is physically and chemically stable for about 1 day or more at a temperature of about 36° C. to about 38° C.

In some embodiments, the present invention is directed to a method of evaluating radiotherapy administered to a subject.

In some embodiments, the method comprises injecting an injectable dosimeter composition into the subject at a site near a site to be treated by the radiotherapy. In some embodiments, the injectable dosimeter composition is the same as or similar to those described elsewhere herein, such as in the “SUMMARY” section.

In some embodiments, the method further comprises administering the radiotherapy to the subject.

In some embodiments, the method further comprises detecting the dosimetric signal in the radiation dosimeter material of the injectable dosimeter composition.

In some embodiments, the injectable dosimeter composition is injected between the site to be treated by the radiotherapy and a site not to be treated by the radiotherapy.

In some embodiments, the method further comprises, while administering the radiotherapy, adjusting the radiotherapy pattern and/or intensity based on the detected dosimetric signal.

In some embodiments, the method further comprises establishing a relationship between a change in the dosimetry signal in the injectable dosimeter and a dose of radiation received by the injectable dosimeter.

In some embodiments, the present invention is directed to a method of performing radiotherapy on a subject.

In some embodiments, the method comprises injecting an injectable dosimeter composition into the subject at a site near a disease site of the subject. In some embodiments, the injectable dosimeter composition is the same as or similar to those described elsewhere herein, such as in the “SUMMARY” section.

In some embodiments, the method further comprises applying radiation to the disease site of the subject.

In some embodiments, the method further comprises detecting the dosimetric signal in the radiation dosimeter material of the injectable dosimeter.

In some embodiments, the injectable dosimeter composition is injected between the disease site and a site not to be treated by the radiotherapy.

In some embodiments, the method further comprises, while applying the radiation, adjusting the radiation pattern and/or intensity based on the detected dosimetric signal.

In some embodiments, the method further comprises establishing a relationship between a change in the dosimetry signal in the injectable dosimeter and a dose of radiation received by the injectable dosimeter.

In some embodiments, the present invention is directed to a method of providing a spacer and/or a marker in a radiotherapy.

In some embodiments, the method comprises injecting an injectable dosimeter composition near the site to be treated by the radiotherapy. In some embodiments, the injectable dosimeter composition is the same as or similar to those described elsewhere herein, such as in the “SUMMARY” section.

In some embodiments, the injectable dosimeter composition is injected as a spacer between the site to be treated by the radiotherapy and a site not to be treated by the radiotherapy, thereby protecting the site not to be treated by the radiotherapy from radiation.

In some embodiments, the injectable dosimeter composition is injected as a spacer to stabilize a site to be treated by the radiotherapy or a site not to be treated by the radiotherapy during the radiotherapy.

In some embodiments, the injectable dosimeter is injected as a spacer that conforms to irregular surface of a site to be treated with radiotherapy.

In some embodiments, the injectable dosimeter is injected as a marker to increase the contrast between a site to be treated by the radiotherapy and a site that is not to be treated by the radiotherapy.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1E illustrate certain aspects of a macromer synthesis and formation of an exemplary hydrogel in accordance with some embodiments. FIG. 1A: Hyaluronic acid polymers were modified with norbornene (HANor). FIG. 1B: Hyaluronic acid polymers were modified with tetrazine groups (HATet). FIG. 1C: HANor and HATet polymers were dissolved rapidly at room temperature and were loaded into syringes for mixing through a Luer-Lock coupler. FIG. 1D shows that the exemplary macromer solution, when mixed, can be injected prior to formation of the exemplary hydrogel and conforms to shape of the space it occupies. FIG. 1E: HANor and HATet spontaneously form stable crosslinked hydrogels when combined.

FIGS. 2A-2C illustrate certain aspects of an exemplary hydrogel dosimeter in accordance with some embodiments. FIG. 2A: Sulfuric acid and a ferrous source are combined with the HANor and HATet pre-hydrogel solution. FIG. 2B: Upon mixing, HANor and HATet form spacer hydrogels with dosimeter ability. FIG. 2C: Ferrous ions are oxidized to ferric ions upon irradiation, and the amount of conversion, which correlates with radiation dose, can be detected through MRI.

FIGS. 3A-3C show preliminary results of an exemplary hydrogel dosimeter in accordance with some embodiments. FIG. 3A: Layout of samples in MRI imaging holder. Samples A-C: HANor-HATet spacer-dosimeter hydrogels with 10 mM, 25 mM, and 50 mM sulfuric acid concentration. Sample D: Standard Fricke gel formulation with 50 mM sulfuric acid. Samples E-N: Standard gelatin Fricke gel formulation. FIG. 3B: Calculated dose (Gy) shown in colorwash over an MRI image. FIG. 3C: Measured dose (a.u.) obtained by the difference in MRI intensity.

FIGS. 4A-4B illustrate some applications of an exemplary hydrogel dosimeter in accordance with some embodiments. FIG. 4A: An in vivo dosimeter separates an organ at risk, e.g., rectum, from the treatment region thereby reducing radiation dose received by critical structures and provides corroboration of dose delivery. FIG. 4B: The in vivo dosimeter compensates for the uneven surface of an ulcerated lesion allowing more uniform dose delivery while also providing corroboration of dose delivery.

FIGS. 5A-5B illustrate certain aspects of the evaluation of several exemplary hydrogel dosimeter in accordance with some embodiments. FIG. 5A: The radiation response of the hydrogel dosimeters with varying concentrations of sulfuric acid were compared with each other and a gelatin-based Fricke dosimeter. FIG. 5B: An exemplary hydrogel sample were manufactured at day 1. The sample was kept at human body temperature and treated with radiation at day 2, day 7 and day 9. The signal intensity differences of the injectable dosimeter compositions measured at multiple time points correspond well to the cumulative dose of the applied radiation over the time course.

FIG. 6 illustrates some aspects of a tissue phantom characterization on a non-limiting sample of the injectable dosimeter, in accordance with some embodiments. A pig muscle tissue was injected with the sample of the injectable dosimeter compositions and applied with radiation. MRI images of the pre-irradiated tissue, the post-irradiated tissue and the difference image were obtained.

FIGS. 7A-7B illustrate turn-on and ratiometric fluorescent probes of Fe³⁺ applicable to live cell imaging.

FIGS. 8A-8C illustrate the cuvette configuration, MRI imaging, and irradiation scheme, in accordance with some embodiments. FIG. 8A: Injectable hydrogel dosimeter Sample 1 (S1) and Sample 2 (S2) are surrounded by gel-containing cuvettes (G) and three additional cuvettes containing mineral oil (M). FIG. 8B show the MRI imaging and irradiation scheme. FIG. 8C is a schematic shows photon beam and depth used to acquire MRI intensity.

FIGS. 9A-9B illustrate the TPS dose, in accordance to some embodiments. (FIG. 9A) Relative isodose distribution and (FIG. 9B) DVH show homogeneous dose distribution of injectable hydrogel dosimeter samples at a depth of 1.2 cm.

FIG. 10 illustrates the change in T1-weighted MRI intensity with MU delivered in a single session to injectable hydrogel dosimeters at room temperature (RT) and body temperature (BT), in accordance with some embodiments. Change in MRI intensity is linear with dose and BT samples show a larger signal change when compared to RT samples.

FIG. 11 illustrates the change in T1-weighted MRI intensity with delivered MU of injectable hydrogel dosimeters across five fractions, in accordance with some embodiments. Injectable hydrogel dosimeters at BT show a decrease in radiation response between Fx1 and Fx2 which then stabilizes from Fx2 to Fx5.

FIGS. 12A-12B illustrate the absolute T1-weighted MR image intensity for RT and BT samples across multiple sessions, in accordance with some embodiments. MRI intensity of RT samples is cumulative across fractions (FIG. 12A), whereas BT samples relaxed to baseline between Fx2 and Fx5 (FIG. 12B).

FIGS. 13A-13B illustrate the absolute T1-weighted image intensity values as a function of cumulative dose for BT injectable hydrogel dosimeters 1- or 2-weeks post-manufacture, in accordance with some embodiments. Injectable hydrogel dosimeters irradiated 1-week (BT 1w) (FIG. 13A) or 2-weeks (BT 2w) (FIG. 13B) post-manufacture show that the initial baseline value differed from BT 0w injectable hydrogel dosimeters but behavior was otherwise similar.

FIG. 14 illustrates the absolute T1-weighted image intensity values as a function of time after a single irradiation, in accordance with some embodiments. An injectable hydrogel dosimeter was measured then irradiated (yellow vertical line) followed by six additional measurements, showing that MRI intensity stabilizes rapidly and relaxes back to baseline within 24 hours.

FIGS. 15A-15D illustrate the dose response of injectable hydrogel dosimeter placed in a soft tissue phantom, in accordance with some embodiments. Injectable hydrogel dosimeter was injected into a pork chop phantom (FIG. 15A) and then imaged before (FIG. 15B) and after (FIG. 15C) irradiation with 600 MU. The annotated image shows that the hydrogel remains at site of injection and emits a signal change post-irradiation (FIG. 15D).

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The study described herein (“the present study”) developed a gel-based dosimeter composition including a polymer gel and a radiation dosimeter material distributed in at least a portion of the polymer gel. Both the polymer gel and the radiation dosimeter material are biocompatible and, as such, the gel-based dosimeter can be injected into the body of a subject. The present study then evaluated the performance of the gel-based dosimeter and found it to have desirable features for use in conjunction with radiotherapy as radiation dosage readouts as well as spacers to protect tissues that should receive no or minimized amounts of radiation.

Accordingly, in some aspects, the instant invention is directed to an injectable dosimeter composition.

In some aspects, the instant invention is directed to a method of evaluating radiotherapy in a subject.

In some aspects, the instant invention is directed to a method of performing radiotherapy in a subject.

In some aspects, the instant invention is directed to a method of providing a spacer and/or a marker in radiotherapy. In certain embodiments, the spacer and/or marker protects healthy tissues from radiation during radiotherapy in a subject.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments±5%, in certain embodiments±1%, in certain embodiments±0.1% from the specified value, as such variations are appropriate to perform the disclosed methods.

### Injectable Dosimeter Composition

In some aspects, the present invention is directed to an injectable dosimeter composition. In some embodiments, the injectable dosimeter composition is biocompatible and, as such, can be injected into the body of a subject without causing substantial adverse effects to the subject.

In some embodiments, the biocompatible injectable dosimeter composition comprises a polymer gel and a radiation dosimeter material distributed in at least a portion of the polymer gel. In some embodiments, the radiation dosimeter material generates a dosimetric signal in response to a form of radiation. In some embodiments, the radiation comprises x-rays, gamma rays, electron beams, protons, or combinations thereof.

As used herein, the term “polymer gel” refers to any gel formed by polymers and/or any mixtures, combinations, or compositions that are able to undergo gelation to form the gels. One of ordinary skill in the art would understand whether the disclosures herein are referring to the gels, the mixtures/combinations/compositions that are able to undergo gelation to form gels, or both, depending on the context.

In some embodiments, the polymer gel is a crosslinked polymer gel. It is desirable that the radiation dosimeter material does not substantially diffuse within the polymer gel, which would cause the dosimetric signal to diffuse away from the location where the radiation is received. In certain embodiments, the crosslinking in the polymer gel is able to limit and/or slowdown the diffusion of the radiation dosimeter material.

In some embodiments, the polymer gel is a hydrogel. As used herein, the term “hydrogel” refers to crosslinked polymer networks of high-water content, such as about 50% weight or more, about 60% weight or more, about 70% weight or more, about 80% weight or more, or about 90% weight or more based on the total weight of the hydrogel. The polymer networks in the hydrogel are often hydrophilic in nature. Hydrogels are sometimes chosen for the injectable dosimeter compositions herein because the dosage of radiation delivered to a site, such as a tissue or the injectable dosimeter, is affected by the presence and content of water in the tissue, and the water content in the hydrogel allows the dosimeter material to detect and measure the radiation with higher accuracy. Non-limiting examples of hydrogels include alginate hydrogels, carboxymethylcellulose hydrogels, collagen hydrogels, hyaluronic acid hydrogels, polyethylene glycol (PEG) hydrogels, poly (2-hydroxyethyl methacrylate) (pHEMA) hydrogels, poly (2-hydroxypropyl methacrylate) hydrogels, polymethylmethacrylate (PMMA) hydrogels, poly-lactic acid (PLA) hydrogels, polyacrylamide hydrogels, derivatives thereof, or combinations thereof. Hydrogels used in the clinic are described in, for example, Mandal et al. (Bioeng Transl Med. 2020; 5:e10158). The entirety of this reference is hereby incorporated herein by reference.

In some embodiments, the polymer gel, such as the hydrogel, is crosslinked. Polymer gels, such as hydrogels, can be crosslinked by various methods.

For example, the polymers can be crosslinked by small-molecule cross-linking, such as by bi-functional molecule (or multi-functional molecule) crosslinkers that interconnect the polymer chains. Non-limiting examples of small-molecule crosslinking include those involving aldehyde and amino groups to form Schiff base. Such crosslinkers include dialdehydes such as glyoxal and glutaraldehyde (which that forms covalent imine bonds with the amino groups, such as amino groups of chitosan, via Schiff reaction). Horseradish peroxidase (HRP) and hydrogen peroxide (H₂O₂) can be used as cross-linkers to crosslink, for example, polymers including tyramine groups. Genipin can be used to crosslink polymers with amino-terminated groups.

For example, the polymers can comprise, or be functionalized with, reactive functional groups, which eliminates the need for a separate crosslinker molecule. In some embodiments, the reactive functional groups are a nucleophile (e.g., an amine group or a thiol group) and a vinyl group, with can undergo a Michael addition to interconnect the polymer chains. In some embodiments, the reactive functional groups are an aldehyde group and a hydrazide group, which can form a hydrazone bond.

For example, in one embodiment, the polymers can comprise, or be functionalized with, a photo cross-linking group. In some embodiments, the photo cross-linking group is an azide group (—N₃), which is converted to nitrene group (R—N:) under the exposure to UV-light and binds to a free amino group. In some embodiments, the photo cross-linking group is an acrylate group (CH₂═CHCOO⁻), which crosslink to other acrylate group when irradiated by UV.

For example, in other embodiments, the polymers can be crosslinked by enzyme-catalyzed cross-linking reaction between the polymer chains. A number of enzymes including transglutaminases (TG), peroxidases, tyrosinase, phosphopantetheinyl transferase, lysyl oxidase, plasma amine oxidase, and phosphatases have been used to form the crosslinking. TG catalyzes the reaction between a free amine group of a protein or peptide-bound lysine and the g-carboxamide group of a protein or peptide-bound glutamine. HRP and soy bean peroxidase are examples of peroxidase enzymes that can catalyze the conjugation of phenol and aniline derivatives in the presence of substrate H₂O₂. Tyrosinases are oxidative enzymes that convert tyrosine residues (e.g. gelatin) into reactive o-quinone moiety, which can react with available nucleophiles such amino groups.

In some embodiments, the injectable dosimeter composition is stable at body temperature of a subject, such as a mammal or a human. In some embodiments, the injectable dosimeter composition is stable at a temperature ranging from about 36° C. to about 38° C. In some embodiments, the injectable dosimeter composition is stable at the above temperature ranges for about 1 day or longer, such as about 2 days or longer, about 3 days or longer, about 5 days or longer, about 7 days or longer, about 10 days or longer, about 15 days or longer, about 20 days or longer, or about 30 days or longer. In some embodiments, the injectable dosimeter composition, such as the polymer gel or the hydrogel therein does not substantially dissolve or otherwise change physical or chemical properties at the temperature ranges over the time periods.

In some embodiments, the polymer gel comprises a crosslinked hyaluronic acid polymer. In some embodiments, the polymer gel comprises a hydrogel formed by reacting a hyaluronic acid polymer modified with a norbornene group (HANor) and a hyaluronic acid polymer modified with a tetrazine group (HATet). As described elsewhere herein, the hydrogels formed by the HANor and HATet are stable for extended time (e.g., at least 8 days) at human body temperature and, when used in conjunction with an exemplary non-limiting transition metal based dosimeter material, produced excellent dosimetric signal at low sulfuric acid concentrations.

In some embodiments, the polymer gel is not gelated. In some embodiments, the polymer gel is partially polymerized. In some embodiments, the polymer gel has not been injected into the body of a subject. One of ordinary skill in the art would understand that polymer gels that have not been fully polymerized, such as have not been crosslinked, have better fluidity and can be easily handled and conveniently injected.

In some embodiments, the polymer gel is gelated. In some embodiments, the polymer gel is fully polymerized. In some embodiments, the polymer gel has been injected into the body of a subject. One of ordinary skill in the art would understand that polymer gels that have gelated or fully polymerized, such as have been crosslinked, have little fluidity and are able to keep the shape inside the body of the subject. The ability to keep the shape is desirable for the injectable dosimeter compositions so as to accurately show the dosage of radiation received at different locations, as well as separate the healthy tissues from disease sites that need to be irradiated. All radiation-sensitive components that do not substantially compromise the biocompatibility of the injectable dosimeter compositions can be used as the radiation dosimeter material. In some embodiments, the radiation-sensitive components are those that can be read via magnetic resonance (MR) or optical imaging. Such radiation-sensitive components are described in, for example, Watanabe et al. (World J Radiol. 2017 Mar. 28; 9 (3): 112-125). The entirety of the reference is hereby incorporated herein by reference.

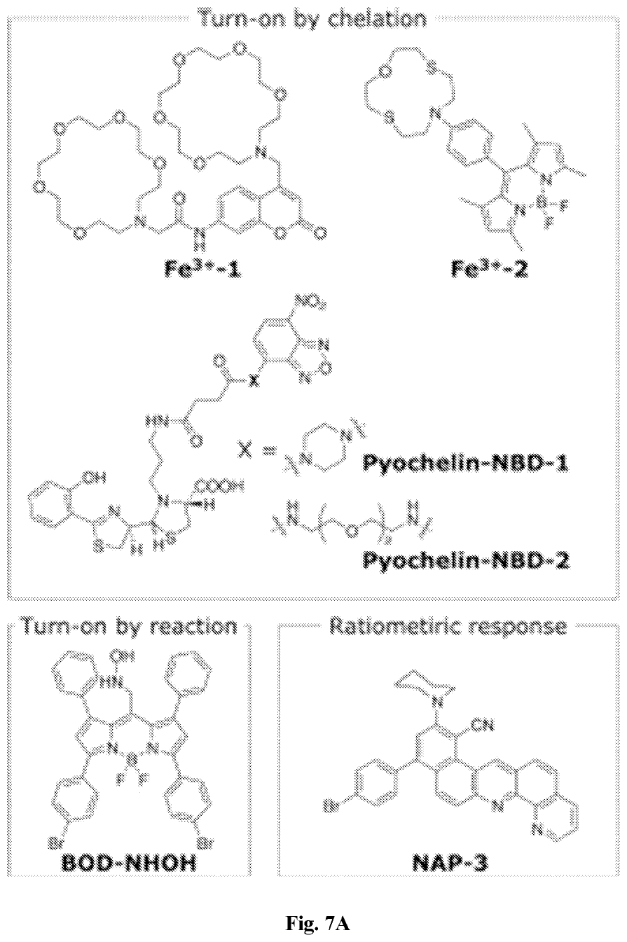

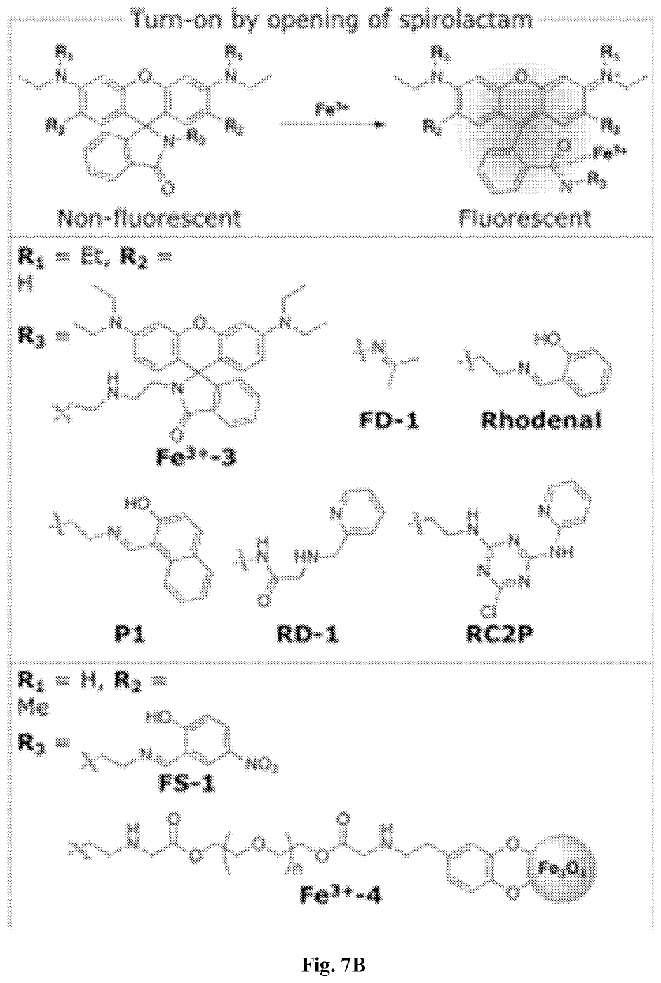

In some embodiments, the radiation dosimeter material comprises a metal element. In some embodiments, the radiation dosimeter material comprises a transition metal element. In some embodiments, the metal element in the injectable dosimeter compositions exists in the form of a metal ion, a metal coordination complex, a metal-containing molecule, or any combinations thereof. In some embodiments, the metal element is included in a nanoparticle. In some embodiments, the metal element includes Fe and/or Gd. In some embodiments, the radiation dosimeter material comprises iron (II), and the radiation converts the iron (II) to iron (III). Iron (II) and iron (III) can be distinguished by both MR and optical imaging methods and quantified and was used as an example to show the feasibility of the injectable dosimeter compositions herein (see e.g., the “Examples” section). One of ordinary skill in the art, reading in light of the instant specification, would know how to match the type of radiation, the dosimeter material, and/or the detection method. For example, one of ordinary skill in the art, reading in light of the instant specification, would understand that, when using optical methods to detect the conversion from iron (II) to iron (III), a chelating agent is sometimes included to aid the detection. Non-limiting examples of such chelating agents are provided in Hirayama & Nagasawa, 2017, J Clin Biochem Nurt 60 (1): 39-48, which is incorporated herein in tis entirety. Non-limiting examples of such chelating agents are further exemplified in FIGS. 7A-7B.

In some embodiments, the radiation dosimeter material comprises a polymer gel dosimeter composition. Examples of polymer gel dosimeter compositions include those using methacrylic as a monomer (such as the methacrylic acid gels or “MAG”) and those using acrylamide as a monomer (such as the polyacrylamide gels or “PAG”).

In some embodiments, the dosimetric signal generated by the radiation dosimeter material in response to the radiation is substantially proportional to the dose of radiation applied to the radiation dosimeter material.

In some embodiments, the dosimetric signal generated by the radiation dosimeter material in response to the radiation is detectable by magnetic resonance imaging (MRI) and/or an optical imaging method.

In some embodiments, the injectable dosimeter, when subjected to repeated irradiations over a time course of about 3 days or longer (such as about 4 days or longer, about 5 days or longer, about 7 days or longer, about 10 days or longer, about 15 days or longer, or about 30 days or longer), is able to generate dosimetric signals corresponding to the intensities of the radiations over the time course.

One of ordinary skill in the art, reading in light of the instant specification, would understand that the injectable dosimeter compositions can be specifically engineered for various applications, such as applications in radiation oncology. For example, the injectable dosimeter compositions can serve as an injectable spacer that creates a gap between target and surrounding tissue, such as the prostate and/or rectum in prostate cancer patients. The injectable dosimeter composition can serve as an injectable filler that could be used, for example, to mechanically stabilize the target region in post-lumpectomy breast cancer patients, which both needs stabilization and may be subjected to radiotherapies. The injectable dosimeter composition can also serve as an injectable fiducial marker to guide patient positioning, or a tissue-equivalent sheet (also referred to as bolus) that conforms to irregular surfaces such as cavities formed by ulcerated lesions that are otherwise too sensitive for a patient to tolerate contact with a harder material. Accordingly, in some embodiments, the gelation time, the Young's modulus, the tensile strength and/or other mechanical properties of the polymer gel are engineered such that the injectable dosimeter composition is tailored to such specific applications.

### Method of Evaluating Radiotherapy

In some aspects, the present invention is directed to a method of evaluating radiotherapy treatment.

In some embodiments, the method comprises injecting an injectable dosimeter composition into the subject at a site near a site to be treated by the radiotherapy; administering the radiotherapy; and detecting the dosimetric signal in the radiation dosimeter material of the injectable dosimeter. One of ordinary skill, reading in light of the instant specification, would understand what is considered “near” the site to be treated by the radiotherapy. In some embodiments, about 100 mm or less is considered “near” for the purpose of this paragraph, such as about 90 mm or less, about 80 mm or less, about 70 mm or less, about 60 mm or less, about 50 mm or less, about 40 mm or less, about 30 mm or less, about 25 mm or less, about 20 mm or less, about 15 mm or less, about 10 mm or less, about 7.5 mm or less, about 5 mm or less, about 4 mm or less, about 3 mm or less, about 2 mm or less, or about 1 mm or less.

In some embodiments, the injectable dosimeter composition is the same as or similar to those described elsewhere herein, such as in the “Injectable Dosimeter” section.

In some embodiments, the injectable dosimeter composition is injected between the site to be treated by the radiotherapy and a site not to be treated by the radiotherapy. The injectable dosimeter composition situated in between separates the to-be-treated area from the not-to-be-treated area.

In some embodiments, the method further comprises adjusting a pattern and/or an intensity of the radiotherapy based on the detected dosimetric signal. In some embodiments, the adjustment is performed in real-time while the radiotherapy is being administered.

In some embodiments, the method further comprises establishing a relationship between a change in the dosimetric signal of the injectable dosimeter composition and a radiation dose received by the injectable dosimeter composition. In some embodiments, the relationship is a linear relationship.

### Method of Performing Radiotherapy

In some aspects, the instant invention is directed to a method of performing radiotherapy.

In some embodiments, the method comprises injecting an injectable dosimeter composition into the subject at a site near a disease site; applying radiation to the disease site; and detecting the dosimetric signal in the radiation dosimeter material of the injectable dosimeter composition. One of ordinary skill, reading in light of the instant specification, would understand what is considered “near” the disease site for a given radiotherapy scenario. In some embodiments, about 100 mm or less is considered “near” for the purpose of this paragraph, such as about 90 mm or less, about 80 mm or less, about 70 mm or less, about 60 mm or less, about 50 mm or less, about 40 mm or less, about 30 mm or less, about 25 mm or less, about 20 mm or less, about 15 mm or less, about 10 mm or less, about 7.5 mm or less, about 5 mm or less, about 4 mm or less, about 3 mm or less, about 2 mm or less, or about 1 mm or less.

In some embodiments, the injectable dosimeter composition is the same as or similar to those described elsewhere herein, such as in the “Injectable Dosimeter” section.

In some embodiments, the injectable dosimeter composition is injected between the disease site and a site not to be treated by the radiotherapy.

In some embodiments, the method further comprises adjusting a pattern and/or an intensity of the radiation based on the detected dosimetric signal. In some embodiments, the adjustment is performed real-time while the radiotherapy is being administered.

In some embodiments, the method further comprises establishing a relationship between a change in the dosimetric signal of the injectable dosimeter composition and a radiation dose received by the injectable dosimeter composition. In some embodiments, the relationship is a linear relationship.

### Method of Providing Spacer or Marker in Radiotherapy

In some aspects, the instant invention is directed to a method of providing a spacer and/or a marker in a radiotherapy.

In some embodiments, the method comprises injecting to the subject an injectable dosimeter composition as the spacer and/or the marker. In some embodiments, the injectable dosimeter composition is the same as or similar to those described elsewhere herein, such as in the “Injectable Dosimeter Composition” section.

In some embodiments, the injectable dosimeter is injected as a spacer between the site to be treated by the radiotherapy and a site not to be treated by the radiotherapy. In some embodiments, the injected injectable dosimeter spaces the disease site from the adjacent healthy tissues, thereby protecting the healthy tissues from radiation during radiotherapy. For example, when treating prostate cancer with radiotherapy, the injectable dosimeter can be injected to space the prostate apart from the rectum, such that the amount of radiation to the rectum is reduced.

In some embodiments, the injectable dosimeter is injected as a spacer to stabilize a site to be treated by the radiotherapy such that the delivery of the radiation is controllable and consistent. In some embodiments, the injectable dosimeter is injected to stabilize a site not to be treated by the radiotherapy such that the avoidance of radiation delivery to the site not to be treated can be achieved or simplified. For example, in a post-lumpectomy radiotherapy that treats breast cancer, the injectable dosimeter can act as a spacer to stabilize target regions for radiation delivery.

In some embodiments, the injectable dosimeter is injected as a spacer that conforms to irregular surface of a site to be treated with radiotherapy. For example, the irregular surface of a cavity, such as the irregular surface a cavity formed by an ulcerated lesion, is a difficult target for radiotherapies. The injectable dosimeter can make the irregular surface both more stable and less irregular in shape and therefore simplifies the radiotherapy.

In some embodiments, the injectable dosimeter is injected as a marker to increase the contrast between a site to be treated by the radiotherapy and a site that is not to be treated by the radiotherapy, thereby simplifying the radiotherapy. One of ordinary skill in the art would understand that the boundary between certain disease tissues and nearby health tissues are sometimes not obvious under imaging techniques. The injected injectable dosimeter can serve as a fiducial marker and simplifies the radiotherapy.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1-1: Self-Forming Hydrogel Chemistry

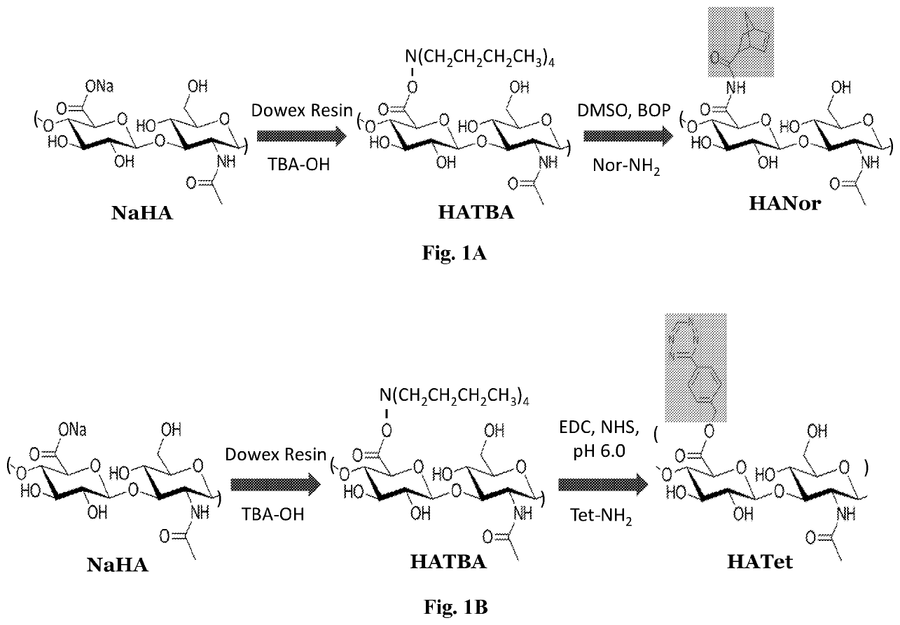

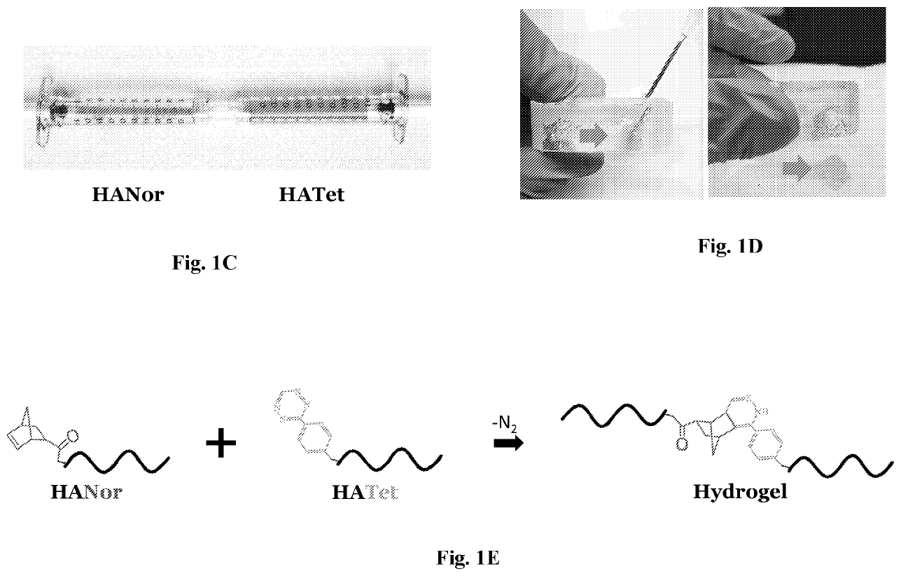

Referring to FIGS. 1A-1E, the present study synthesized a hyaluronic acid polymer-based hydrogel as a non-limiting exemplary polymer gel.

Referring to FIGS. 1A-1B, hyaluronic acid polymers were modified with either norbornene (HANor) (FIG. 1A) or tetrazine groups (HATet) (FIG. 1B). Referring to FIG. 1C, the result HANor and HATet polymers can be dissolved rapidly at room temperature and loaded into syringes for mixing through a Luer-Lock coupler. The present study confirmed that the mixed macromer solution can be injected prior to hydrogel formation and conforms to shape of the space the mixture occupies (FIG. 1D) before the two modified HA polymers spontaneously form stable crosslinked hydrogels (FIG. 1E).

### Example 1-2: Hydrogel-Based Injectable Dosimeter

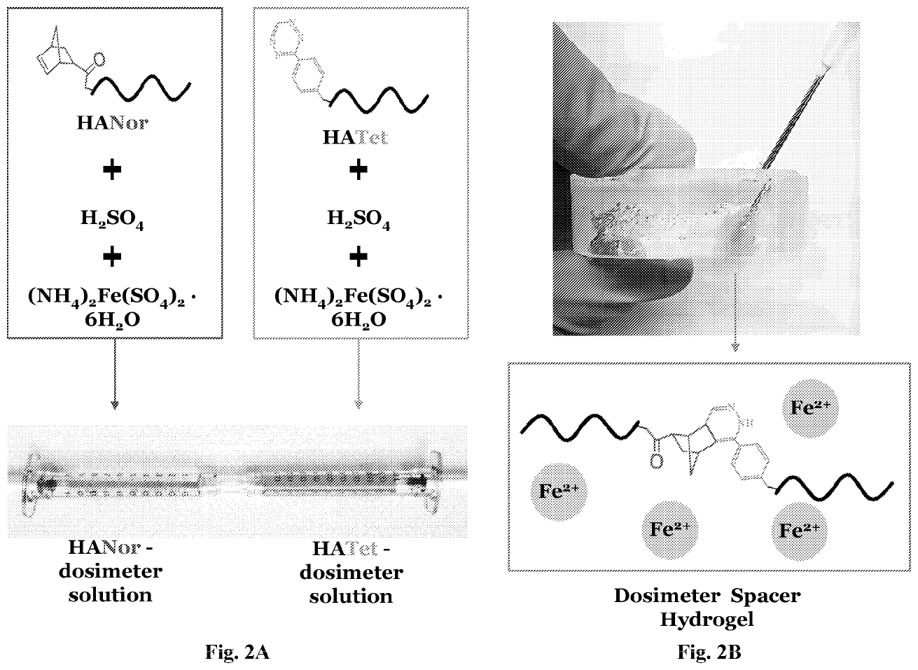

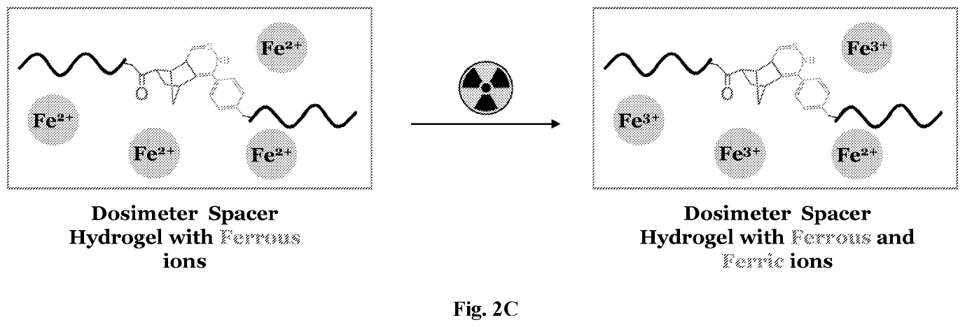

Referring to FIGS. 2A-2C, the present study constructed a non-limiting exemplary injectable dosimeter composition using the hydrogel of Example 1.

Referring to FIG. 2A, sulfuric acid and a ferrous source were combined with the HANor and HATet before the two hydrogel components were mixed. Referring to FIG. 2B, upon mixing, the HANor and HATet form spacer hydrogels with dosimeter ability. Referring to FIG. 2C, ferrous ions are oxidized to ferric ions upon irradiation and the amount of conversion, which correlates with radiation dose, is detectable through MRI.

### Example 1-3: Evaluation of Hydrogel-Based Injectable Dosimeter

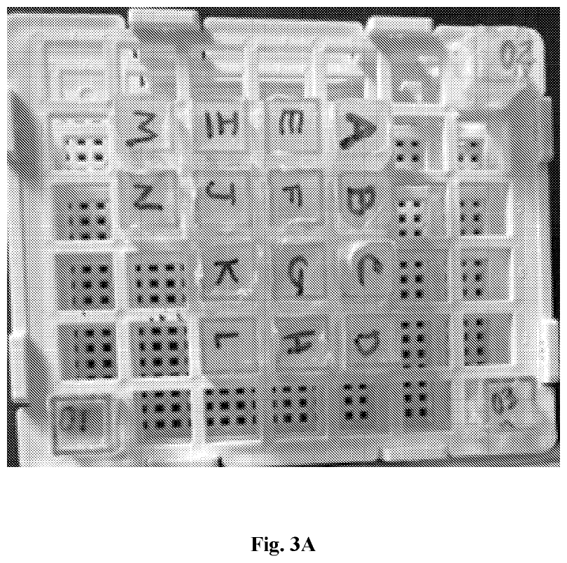

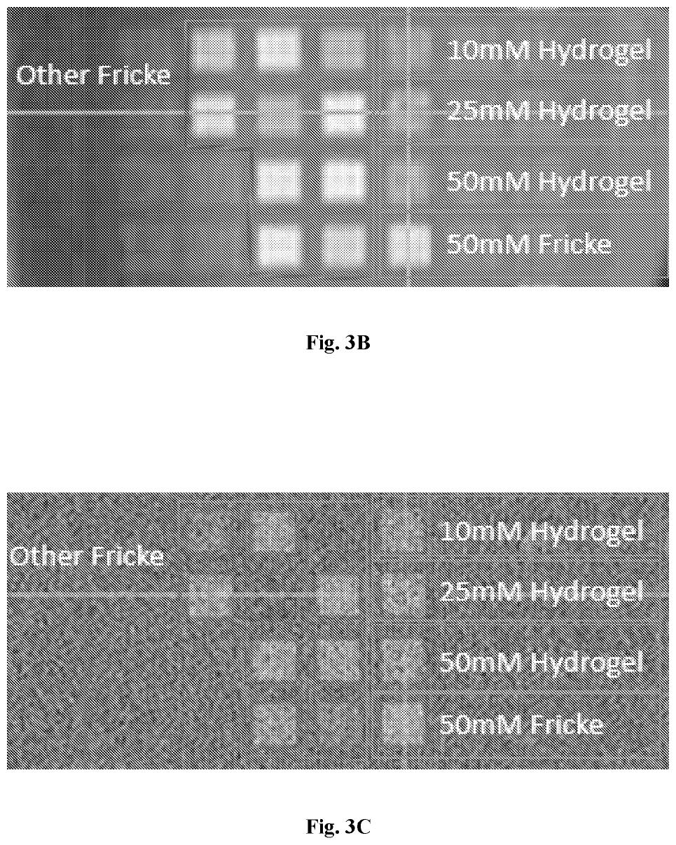

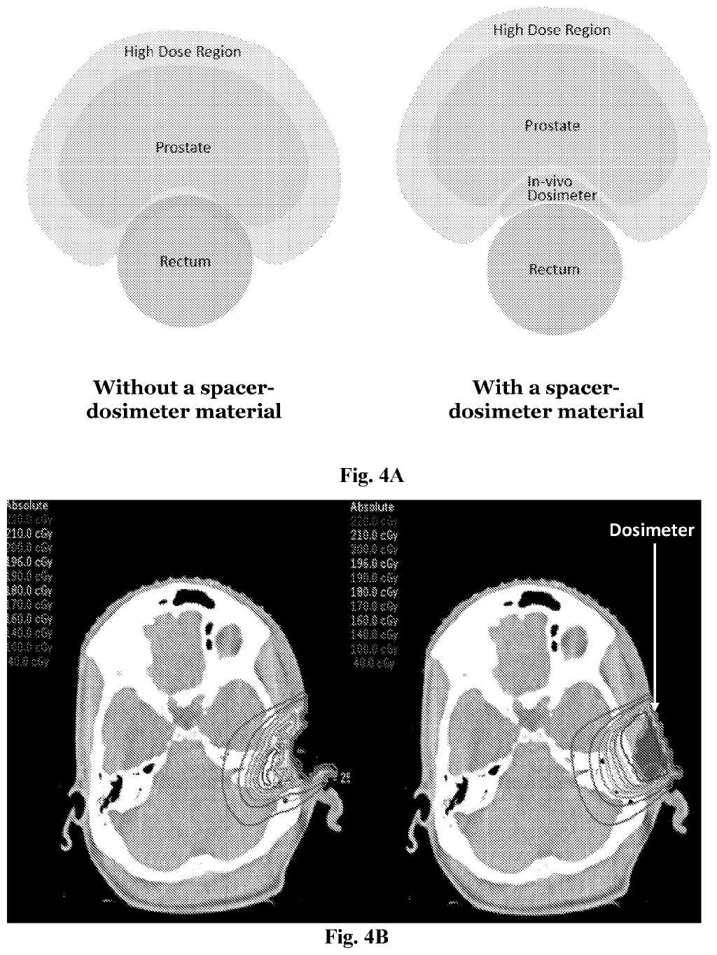

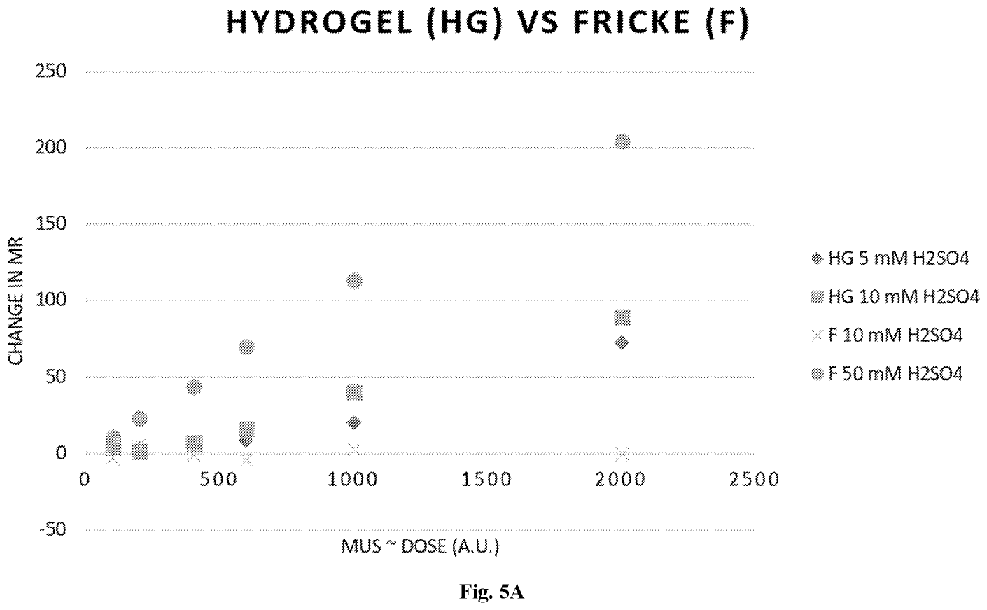

Referring to FIGS. 3A-3C, several non-limiting exemplary injectable dosimeters according to Example 2 were constructed with different sulfuric acid concentration and extruded onto cuvettes. The exemplary injectable dosimeters, as well as the non-injectable gelatin-based Fricke gels, were subjected to x-ray radiations, and the responses of the gels were observed.

Referring to FIG. 3A gel samples were placed in an MRI imaging holder. Samples A-C were injectable dosimeter hydrogels according to Example 2, with 10 mM, 25 mM, and 50 mM sulfuric acid concentrations, respectively. Sample D is a conventional Fricke gel formulation with 50 mM sulfuric acid. Samples E-N are Fricke gel formulations, as well. Referring to FIG. 3B, calculated dose (Gy) the gels were subjected to are shown in colorwash over an MRI image. FIG. 3C shows the measured dose (a.u.) as obtained by the difference in MRI intensity.

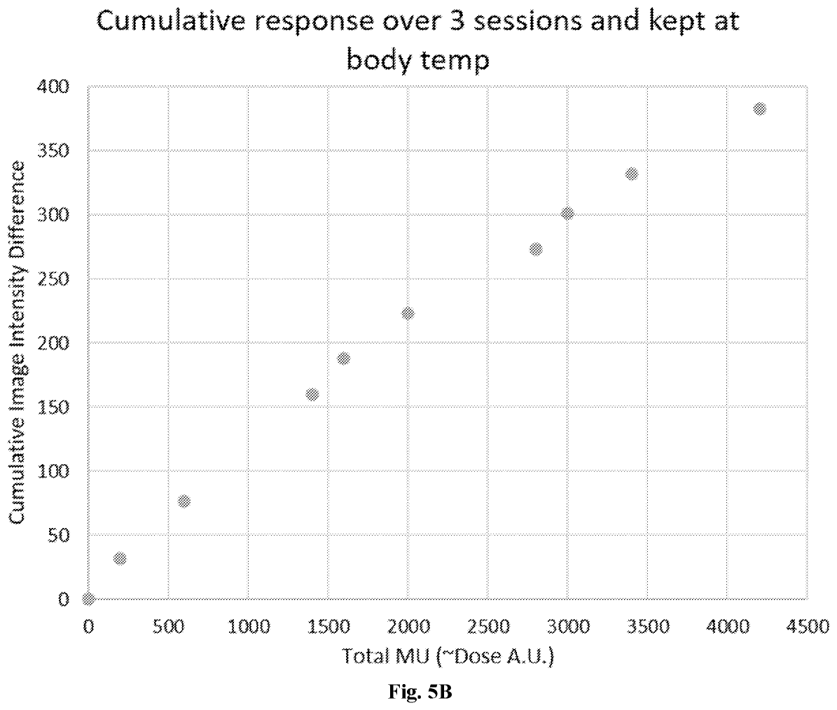

Referring to FIG. 5A, the present study measured radiation response of several gel formulations according to Example 2 by varying the gel percentage and concentration of sulfuric acid. The responses of the injectable dosimeter compositions to radiation were compared to the non-injectable gelatin-based Fricke dosimeters. The results show that, for a given concentration of sulfuric acid, the radiation response of the non-limiting injectable dosimeter compositions according to Example 2 appeared superior to standard Fricke gels such that clinically usable radiation responses were obtainable using relatively lower concentrations of sulfuric acid. Since high sulfuric acid concentrations affect biocompatibility, the superior radiation responses of the injectable dosimeter compositions are desirable for in vivo applications.

Since the radiation response of the non-injectable Fricke gel dosimeter is known to be temperature and time-sensitive, the present study evaluated the radiation responses of an injectable dosimeter composition according to Example 2 for stability. Specifically, the present study constructed the sample of the injectable dosimeter composition at day 1, and kept the sample at human body temperature and applied radiation to the sample on day 2, day 7 and day 9. The cumulative intensity differences were recorded. Referring to FIG. 5B, the signal intensity differences of the injectable dosimeter composition correspond well to the cumulative dose of the applied radiation over the time course. As such, the exemplary injectable hydrogel showed good, clinically usable response at body temperature under repeated irradiation and across varying start times.

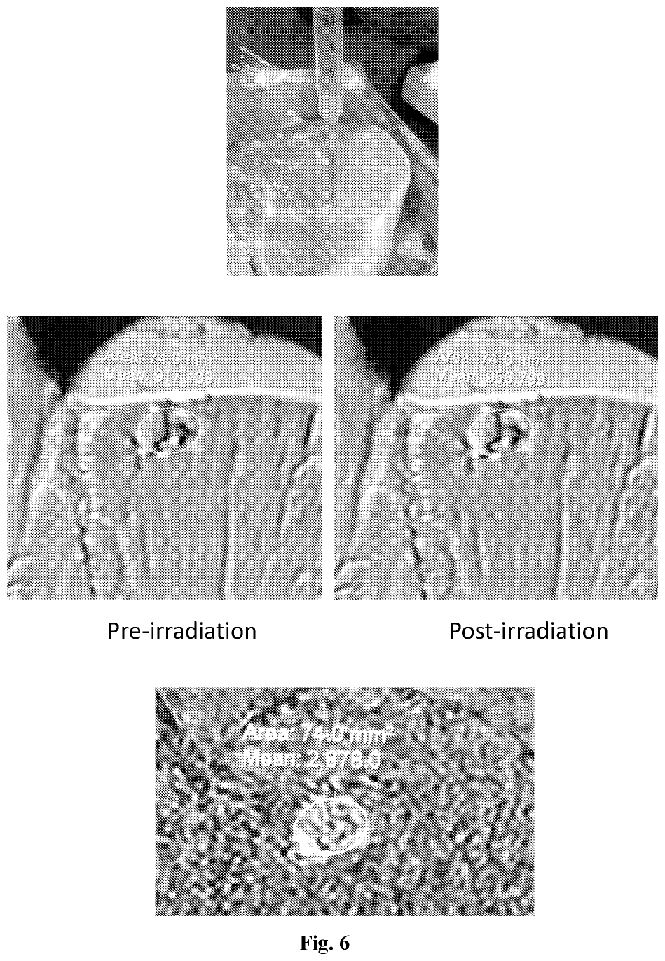

Referring to FIG. 6, the present study performed tissue phantom characterization on a non-limiting sample of the injectable dosimeter compositions of Example 2. Specifically, a pig muscle tissue was injected with the sample of the injectable dosimeter compositions and applied with radiation. MRI images of the pre-irradiated tissue, the post-irradiated tissue and the difference image were obtained.

### Example 2: An Injectable Dosimeter for Real-Time, In Vivo Verification of MR-Guided Radiation Therapy

In vivo dosimetry has long been recognized as a valuable adjunct to radiation therapy for its ability to report the actual dose received by patients. However, it is currently limited to surface and intracavitary measurements, leaving most tumor locations and organs-at-risk inaccessible to dosimetric verification.

Combining work in patient-injectable hydrogels, gel dosimetry with MRI readout, and systems that integrate MRI and radiotherapy, this study reports the development of an injectable hydrogel dosimeter with the possibility of real-time, in vivo patient dosimetry for MR-guided radiation therapy (MRgRT). The injectable dosimeter incorporates Fricke dosimetry components within norbornene- and tetrazine-modified hyaluronic acid macromers that upon mixing self-form into a hydrogel. The present study characterized the system at body temperature over multiple irradiation sessions with clinically relevant injection-to-irradiation time intervals to evaluate radiation responses under conditions relevant to in vivo dosimetry.

Injectable hydrogel dosimeters were extruded into cuvettes, sealed, protected from light, and stored at room or body temperature. At zero, one, or two weeks post-manufacture, samples were irradiated to 16.8 Gy on a 1.5-T MR-Linac with a 7×FFF beam. T1-weighted 3D Fast Field Echo MR images (TR/TE=11/3.8 ms, 8 signal averages) were obtained before, after, and twice during irradiation with the beam off. The average image intensity of the injectable hydrogel dosimeter at a fixed hydrogel depth (1.2 cm) was recorded at the MRI console. This was repeated weekly for a cumulative dose of 84 Gy over five fractions over five weeks. Short-term signal evolution was evaluated by acquiring multiple MR images of a sample up to 72 minutes post-irradiation and again at 24 hours. To demonstrate the use of the injectable hydrogel dosimeter in tissue, a porcine tissue phantom was injected and immediately irradiated and imaged. The pre- and post-irradiation difference image was assessed for signal change at the location of the injected hydrogel dosimeter.

The intra-fraction changes in T1-weighted image intensity of injectable hydrogel dosimeters were linear with radiation dose. Injectable hydrogel dosimeters maintained at body temperature were about twice as sensitive as those at room temperature. For all samples, dose response decreased between the first and second fractions and was stable afterwards. At body temperature, the dose response was consistent and unaffected by manufacture-to-first fraction time. The signal in body temperature samples returned to baseline intensity within 24 hours, and the room temperature samples exhibited no signal clearing. A proof-of-concept porcine tissue phantom study also showed that the injectable hydrogel dosimeter localizes within soft tissue and exhibits a dose response post-irradiation.

Here, a novel, MR-readable injectable dosimeter was developed with characteristics suitable for real-time in vivo dosimetry for MRgRT. The injectable hydrogel dosimeter can measure clinically relevant dose levels for multiple irradiations over several weeks.

### Example 2-1

Twenty years ago, Oldham et al. observed, “rapid advances in the technology to deliver radiation treatments have not been paralleled by corresponding advances in the ability to verify these treatments” (Med Phys. 2003; 30 (4):623-634). Since then, while patient-specific quality assurance (PSQA) via pretreatment phantom measurements has become routine practice, in vivo dosimetry, the measurement of radiation dose received by the patient during treatment, has in contrast seen less advancement. This is despite the fact that PSQA is unable to detect a number of treatment errors where in vivo dosimetry could be used to avoid mistreatment.

Despite its acknowledged importance, the use of in vivo dosimetry has been limited. By definition, an in vivo dosimeter must be placed in the patient and, in almost all cases, the dosimeter must also be either connected to a readout device or recovered for readout after radiation. Almost all available in vivo dosimeters (e.g., diodes, TLDs, MOSFETs, OSLDs, plastic scintillation detectors, film) are therefore limited to point-dose measurements at surface or intracavitary locations, leaving most tumor locations and organs-at-risk inaccessible to dosimetric verification. Implantable MOSFETs with RF telemetric readout have been investigated to address this limitation. However, for a number of reasons such as invasive and permanent implantation, measurement uncertainty due to dependencies on orientation, energy, and drift, and an inability to perform real-time, non-point dosimetry, implantable MOSFETs were not widely used and are currently not available.

Since that time three key developments in implantable biomaterials and in the field of radiation therapy provide opportunities for more versatile in vivo dosimeters. First, injectable hydrogels are now widely used as rectal spacers, and other applications for injectable hydrogels have been reported including their use as a tissue marker for image guidance and target definition, and as a radiation-responsive drug delivery system. Second, magnetic resonance-guided radiation therapy (MRgRT) systems are now commercially available and their clinical use is on the rise. Third, the availability of MRgRT systems has led to a revived interest in using MR-readable Fricke and polymer gel dosimeters for real-time, volumetric dose measurements. The combination of these developments suggests the possibility of developing an injectable hydrogel dosimeter that could provide real-time, volumetric in vivo dosimetric information during treatment with an MRgRT device. Such a dosimeter could offer numerous potential benefits. The efficacy of organ-at-risk (OAR) sparing could be assessed if the injectable hydrogel dosimeter were used as a geometric spacer, such as between the prostate and rectum. Target dose could be assessed directly if the injectable hydrogel dosimeter were placed at or in a target such as a lumpectomy cavity. Placed at a target/OAR interface, the real-time dosimetric effect should the OAR move into the radiation beam due to intrafraction motion could be evaluated. In short, the promise of in vivo dosimetry to “catch treatment errors, assist in treatment adaptation, and record the actual dose delivered to the patient,” could be more fully realized.

The present study reports the development of a novel dosimeter that combines Fricke gel dosimetry with an injectable, self-forming hydrogel system. Fricke gels can be read using MRI because their longitudinal (spin-lattice) relaxation time T1 changes proportionally with radiation dose and, in view of an injectable in vivo application, were selected over polymerizing dosimeters because of toxicity issues associated with the latter. Fricke gel dosimeters have already been used to characterize MRgRT systems, exploiting the capability of the MR-Linac to interrogate the dosimeter immediately after irradiation without requiring that it be moved to another device. Notably, Lee et al. has used Fricke gels to measure radiation deposition in real-time using fast imaging of the dosimeter during radiation delivery.

The injectable hydrogel dosimeter chemistry exemplified in this study relies on an irreversible Diels Alder reaction between dienophiles and dienes (see e.g., Alge et al., Biomacromolecules. 2013; 14(4):949-953). Here, hyaluronic acid (HA) macromers were modified with norbornene (dienophile) or tetrazine (diene) to form HANor and HATet, respectively. These macromers can be easily dissolved in water or saline, and upon mixing undergo a Diels Alder reaction resulting in the formation of a hydrogel. Properties such as stiffness and gelation time can be tuned by changing the total macromer concentration and the ratio between HANor and HATet macromers (see e.g., Gultian et al., Macromol Biosci. Published online Dec. 9, 2022:2200425. doi: 10.1002/MABI.202200425). To form injectable hydrogel dosimeters, HANor and HATet were separately dissolved in a phosphate-buffered saline (PBS) solution (1% weight by volume) containing Fricke dosimetry components (1 mM ammonium iron (II) sulfate hexahydrate and 10 mM sulfuric acid). These solutions were then loaded onto separate syringes, mixed with a Luer-Lock coupler, and injected through a syringe needle just prior to gelation.

Using this formulation, the present study reports several characterization tests describing the radiation response of the injectable hydrogel dosimeters under conditions relevant to clinical in vivo dosimetric applications, namely:
1 Radiation response at body temperature (to reflect injected conditions).
2. Radiation response over multiple irradiation sessions (to reflect fractionated delivery).
3 Radiation response as a function of time post-manufacture (to reflect the simulation-to-treatment time interval).
4. MRI signal evolution post-irradiation.

As a final proof-of-concept demonstration, the present study injected the hydrogel dosimeters according to some embodiments into a soft tissue phantom (a pork chop) which was then irradiated and imaged on a 1.5-T MR-Linac.

### Example 2-2

### Setup, Irradiation, Imaging, and Analysis Procedure

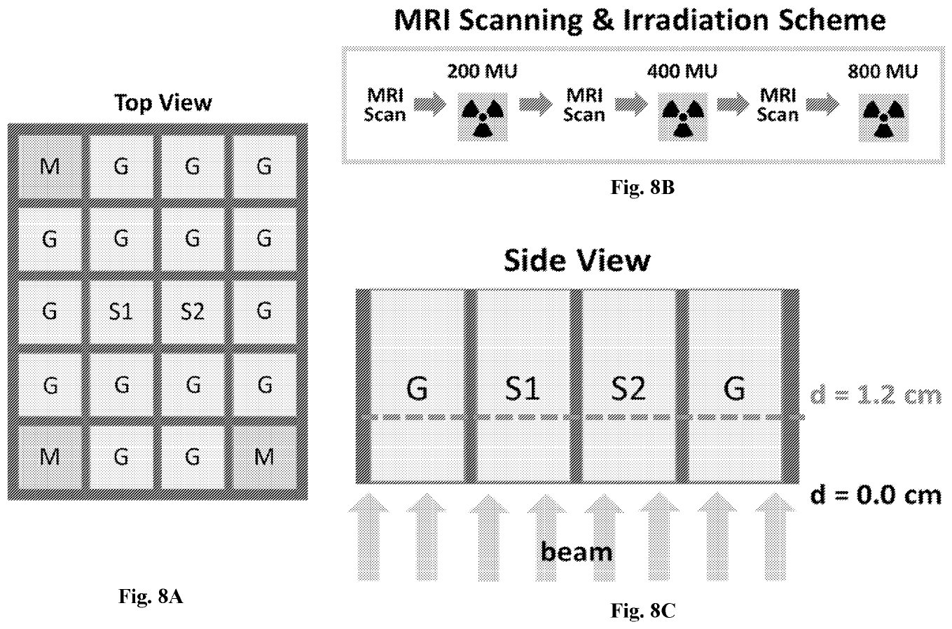

Eight spectrophotometer cuvettes (Supelco, Bellefonte, PA) were each filled with 3 mL of injectable hydrogel dosimeter. The tops of the cuvettes were capped and wrapped with Parafilm to prevent hydrogels from drying out. Cuvettes were protected from light and were kept at room temperature (n=2) or in an incubator at body temperature (37° C.) (n=6). All irradiation and imaging sessions were conducted as follows. Injectable hydrogel dosimeter-containing cuvettes (n=2) were placed in the center of a 5×4 array of cuvettes (FIG. 8A). The surrounding 18 cuvettes were used to provide a uniform scattering medium and contained gelatin gels (G) or mineral oil (M). These remained in the same position for the duration of this study. The mineral oil used in three corner cuvettes was used to provide an image intensity baseline for MRI.

The cuvettes were irradiated using a 1.5-T MR-Linac (Unity, Elekta Inc., Stockholm, Sweden). The cuvette array was positioned using the treatment couch index bar and sagittal laser for reproducibility such that a beam at gantry angle 180° was centered on the two experimental samples. In this geometry, a pre-irradiation MRI was acquired, and then a 10×10 cm² 7-MV flattening filter-free photon beam was delivered three times using 200, 400, and 800 Monitor Units (MUs). MRI scans were acquired immediately after each irradiation (FIG. 8B). The 4 MRI scans were done within a single dynamic session to avoid intra-session image rescaling and used a T1-weighted 3D Fast Field Echo (FFE) sequence with TR/TE=11/3.8 ms and number of signal averages (NSA, also known as number of excitations (NEX)) set at 8. The reconstructed voxel size was 0.3×0.3×1.0 mm³. Each MRI scan took 3 minutes for a field-of-view (FOV) of 86×86×50 mm³ and total irradiation time was about 3.5 minutes resulting in a total of 15.5 minutes of irradiation and imaging time. To acquire T1-weighted MR image intensity values (AU), the region of interest (ROI) tool on the MRI console (Marlin, Philips Healthcare, Amsterdam, Netherlands) was used. The ROIs were drawn in the cuvette at a depth of 1.2 cm from the surface closest to the treatment couch (FIG. 8C). The change in MR image intensity was determined by subtracting the T1-weighted values in the ROI's before and after irradiation.

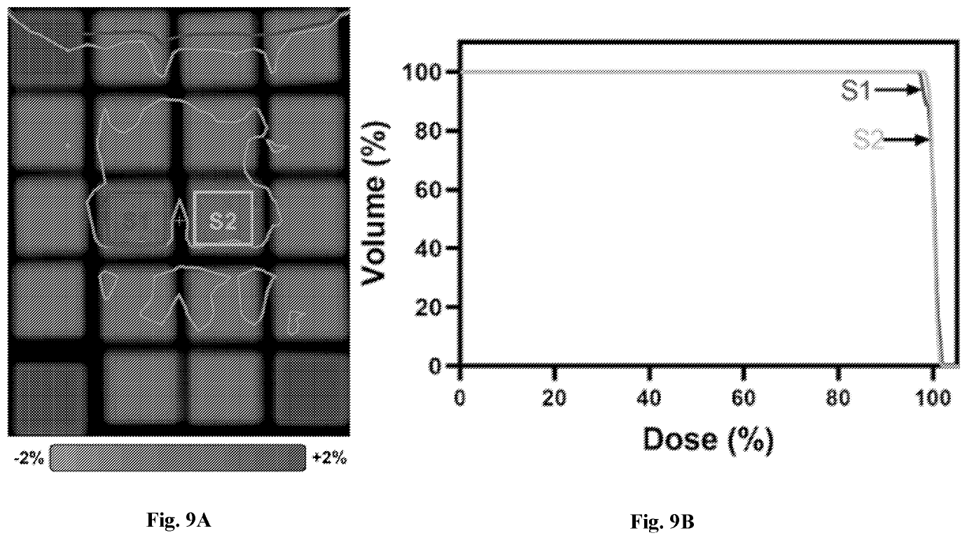

The MRI intensity values were compared to the treatment planning system (TPS) dose (FIG. 9A). At this depth and treatment distance, 200 MU corresponds to 2.4 Gy, and dose was homogeneous at +2% in the injectable dosimeter samples. Dose homogeneity was further confirmed with a dose volume histogram (DVH) (FIG. 9B).

### Irradiation Sessions

Injectable hydrogel dosimeters were subjected to three different clinically relevant scenarios: (1) body temperature (BT) versus room temperature (RT), (2) radiation response over multiple irradiation sessions, and (3) radiation response dependence as a function of time post-manufacture. Cuvettes of injectable hydrogel dosimeter at RT (n=2) and BT (n=2) were imaged and irradiated 1 day post-manufacture as described in Section 2.1. After the initial irradiation session, BT and RT cuvettes were maintained at their corresponding temperatures while avoiding light. For the next four weeks, injectable hydrogel dosimeters in the cuvettes had weekly irradiation sessions using the same setup and irradiation scheme described in FIG. 8B for a total of five irradiation sessions and a cumulative dose of 84 Gy (7000 MU). To evaluate radiation response as a function of time post-manufacture, the remaining BT cuvettes (n=4) followed the same weekly irradiation schedule described above with the initial session taking place one week (n=2) and two weeks (n=2) post-manufacture to reflect potential simulation-to-treatment time intervals. Table 1 below summarizes the irradiation schedule for all samples described in this section.

TABLE 1
Irradiation schedule for RT and BT injectable hydrogel dosimeter
samples. Room temperature (RT) and body temperature (BT) samples
were irradiated over five weekly fractions (F × 1 to F ×
5) starting 0-, 1-, or 2-weeks (0 w, 1 w, 2 w) post-manufacture.

| | Weeks post-manufacture | | | | | | |
|---|---|---|---|---|---|---|---|
| | 1 | 2 | 3 | 4 | 5 | 6 | 7 |
| RT 0 w | F × 1 | F × 2 | F × 3 | F × 4 | F × 5 | | |
| BT 0 w | F × 1 | F × 2 | F × 3 | F × 4 | F × 5 | | |
| BT 1 w | | F × 1 | F × 2 | F × 3 | F × 4 | F × 5 | |
| BT 2 w | | | F × 1 | F × 2 | F × 3 | F × 4 | F × 5 |

### MRI Signal Evolution Post-Irradiation

To evaluate short-term, post-irradiation signal change, a body temperature cuvette was irradiated 4 weeks post-manufacture with 400 MU in the same setup as described in FIG. 8B. 6 scans were acquired pre-irradiation and at 0, 8-, 21-, 39-, 72-, and 1440-minutes (24 hours) post-irradiation.

### Tissue Phantom Trial

To demonstrate proof-of-concept, a pork chop was injected with 300 μL of the hydrogel dosimeter and immediately scanned using the same sequence described in the “Setup, irradiation, imaging, and analysis procedure” section, irradiated with a PA beam to 600 MU, and re-scanned. Marlin software was used to create an ROI around the injectable hydrogel dosimeter and to determine the MRI intensity value pre- and post-irradiation.

### Example 2-3: Injectable Dosimeters at Body Temperature are More Sensitive than at Room Temperature

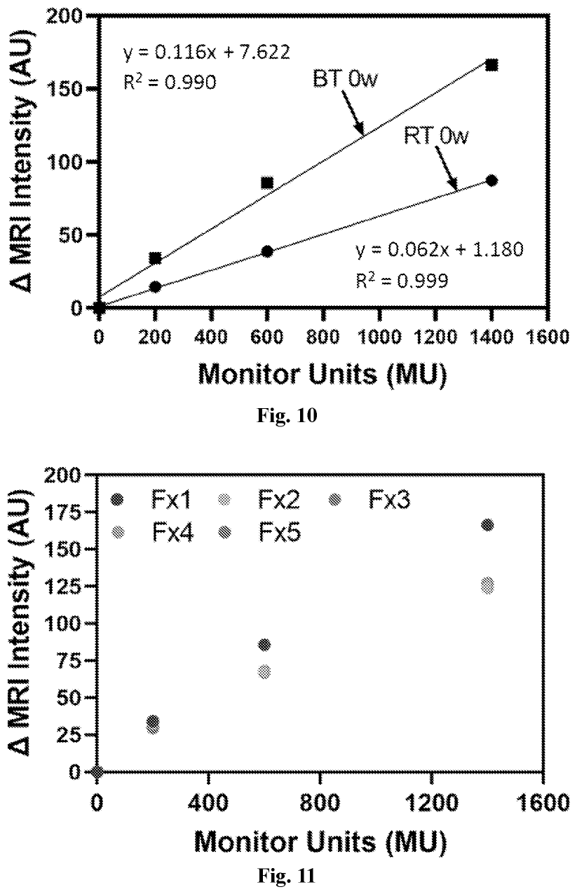

The response of MR-based Fricke gel dosimeters are known to be dependent on temperature. Welch et al. (Phys Med Biol. 2017; 62(8):3221-3236) modeled R₁-temperature dependence of Fricke-xylenol orange dosimeters from 15 to 22° C., reflecting the range between refrigeration to facilitate gelation and its usage at room temperature. Because of the intended in vivo application, the present study sought to evaluate the signal sensitivity between RT and BT injectable hydrogel dosimeter samples. The dose response of the injectable hydrogel dosimeters kept at 37° C., the temperature of its intended application as an in vivo dosimeter, was almost twice that of the room temperature controls, with a linear fit slope of 0.116 compared to 0.062. (FIG. 10).

### Example 2-4: Radiation Response Decreases Between First and Second Irradiation Prior to Stabilizing

Next, the present study were interested in evaluating the stability of the injectable hydrogel dosimeter response across multiple fractions (Fx) at BT, as this will affect how it can be used over a patient treatment course. Comparing dose response across five weekly fractions it was found that injectable hydrogel dosimeter dose response decreased between the first and second fractions (Fx1 versus Fx2) and was stable afterwards (FIG. 11). This finding suggests the first fraction cannot be used for calibration, but also that each session may not need to be separately calibrated.

### Example 2-5: BT Injectable Hydrogel Dosimeter Readouts Reset to Baseline Between Weekly Irradiation Sessions

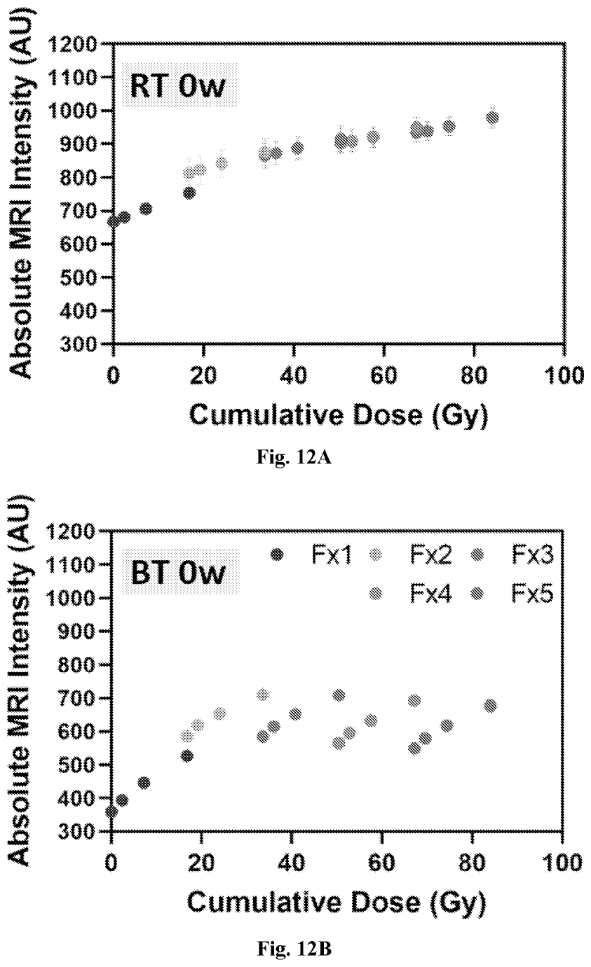

When looking at absolute T1-weighted MRI intensity values instead of relative change, it was observed that between fractions 2 to 5, the signal of the injectable dosimeter kept at body temperature (BT) returned to a baseline (FIGS. 12A-12B). This was not observed in the injectable hydrogel dosimeter kept at room temperature (RT). This finding has implications for the ability of the dosimeter to directly measure fractional versus cumulative dose over a treatment course. Drift in the absolute T1-weighted image intensity value was observed in both samples, most notably between fractions 1 and 2.

### Example 2-6: Relative Radiation Response of the Injectable Hydrogel Dosimeter is Unaffected by Time Post-Manufacture

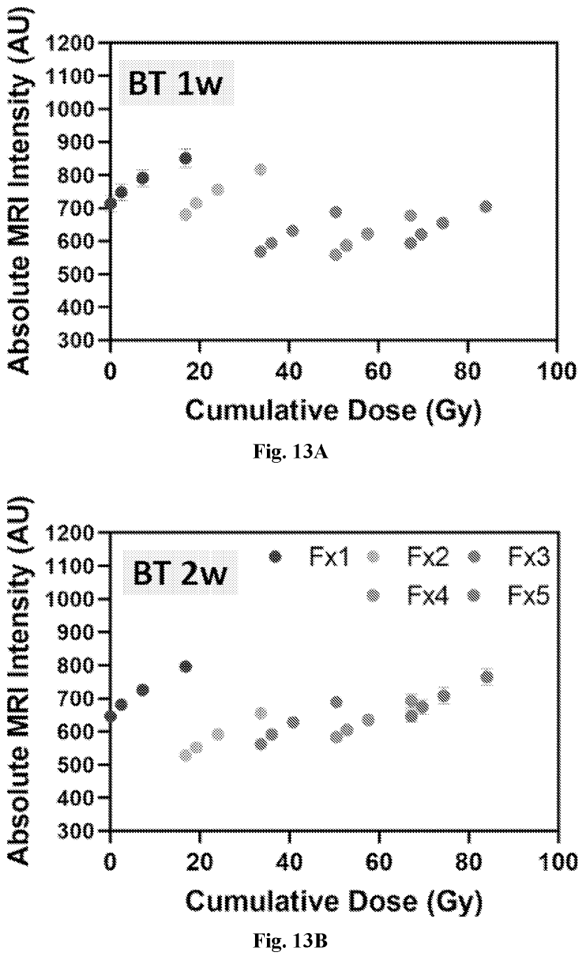

Next, the present study were interested in evaluating the stability of injectable hydrogel dosimeter response as a function of the time interval between injection and irradiation. In its intended application, the dosimeter could be injected at or before the time of simulation, which could be up to a few weeks before irradiation. In addition, having observed the change in response between fractions 1 and 2 (FIG. 11), this part of the study could shed light on whether this change is attributable to radiation or time. The body temperature cuvettes in which initial irradiation was delayed by 1 week (BT 1w) and 2 weeks (BT 2w) exhibited the same behaviors observed for BT 0w samples: (1) stable but decreased radiation response after the first fraction, (2) signal clearing between weekly imaging sessions, and (3) presence of baseline drift though the timing, frequency, and magnitude of the drift differ (FIGS. 13A-13B).

### Example 2-7: MRI Signal Evolution Post-Irradiation

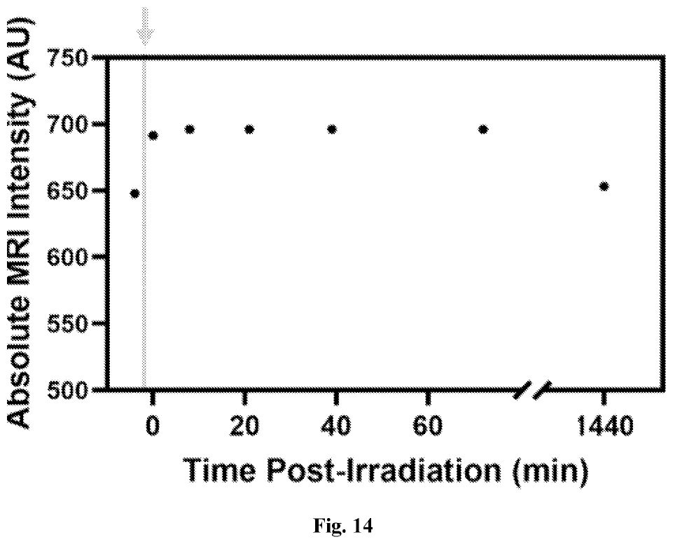

Next, the present study were interested in investigating the MRI signal evolution immediately following irradiation. A cuvette containing the injectable hydrogel dosimeter was measured then irradiated with 400 MU followed by six measurements at different time intervals (FIG. 14). The change in MRI signal from the pre-irradiation baseline took place immediately post-irradiation and stabilized within 8 minutes after irradiation. The signal reverted to baseline within 24 hours. The observation that the dosimeters at body temperature appear to reset within 24 hours means this formulation can be used for fractional dose measurements.

### Example 2-8: Tissue Phantom Trial

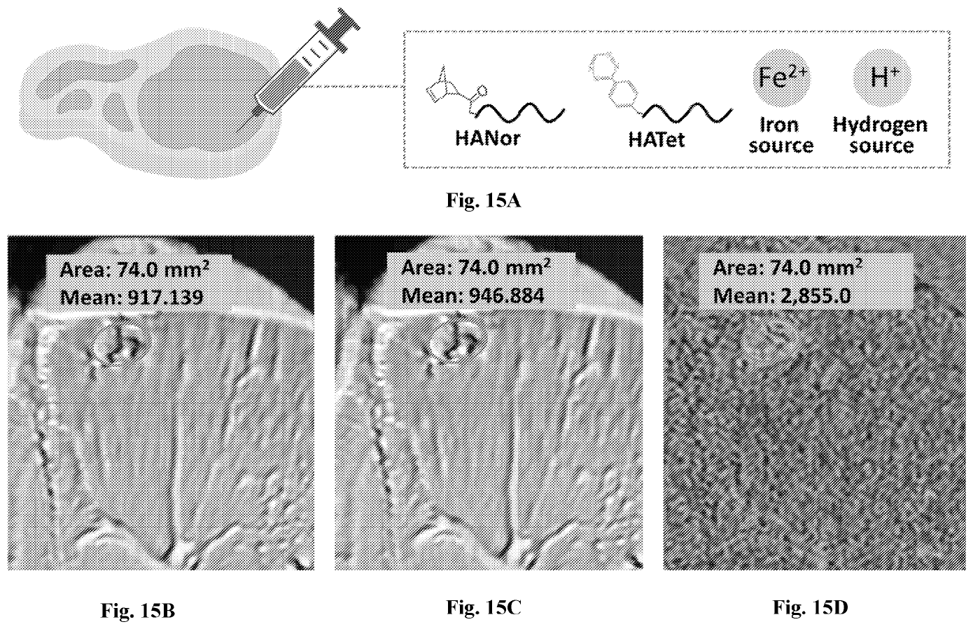

As a final proof-of-concept demonstration, the injectable hydrogel dosimeter was delivered to a soft tissue phantom which was then irradiated and imaged on a 1.5-T MR-Linac (FIGS. 15A-15D). A pork chop phantom was injected with the injectable hydrogel dosimeter (FIG. 15A) and imaged before and after irradiation (FIGS. 15B-15C). The images show the injectable hydrogel dosimeter remains at the injection site and also show an intensity change after irradiation.

### Example 2-9: Modifications from the Injectable Hydrogel Dosimeter's Formulation

The current hydrogel formulation already exhibits the required properties for its intended application as an injectable hydrogel dosimeter: injectability, self-forming, and a linear and reliable radiation response with MR-readable change in T1-weighted signal proportional to the dose delivered, independent of the size of the radiation dose. The present study further identifies three areas that can be modified from the non-limiting example of the injectable hydrogel dosimeter's formulation above.
1. A higher molecular weight hydrogel formulation might be desirable to better support applications such as injection into a cavity (e.g., lumpectomy cavity) or acting as a spacer between organs.
2. A formulation that can retain its signal to allow cumulative dose measurements can be developed.
3. Further biocompatibility study involving injection into a cell, tissue, or animal model can be performed. Such a study can confirm the duration of presence and performance of the gel under in vivo conditions.

While the injectable hydrogel dosimeter was developed with the intention of on-line imaging during an irradiation session as is practical with an MR-Linac, the system could be used with irradiations performed using other devices followed by imaging on a conventional MRI. Therefore, though currently available MR-Linac systems offer a single modality, energy, and dose rate, broader characterization of the dosimeter may be desirable to understand effects of dose rate, dose fractionation, and beam energy on response.

Additional characterization of the injectable hydrogel dosimeter would be facilitated by the availability on the Unity MR-Linac of T1 mapping, which is the standard method for quantitative Fricke dosimetry. T1-weighted imaging of Fricke gel dosimeters with the Unity poses challenges, because each imaging study is separately and automatically scaled by the device. This complicates inter-session comparison and clinical application (e.g., calibration and cumulative measurements). The present study tried to minimize this in the experiments herein by reproducing setup conditions as closely as possible for each scan in the hopes that all of the data would have the same or sufficiently similar scaling. The success in this herein was estimated by looking at the image intensity value of the mineral oil cuvettes in the setup across different sessions and experimental setups. The contents of these cuvettes were radio-insensitive and their image intensity should have been constant throughout the experiments. The actual variation was found to be about 2%. The available T1-weighted imaging technique on the Unity also appeared to introduce slice position-dependent variation in the T1-weighted image intensities, which is why the analysis herein focused on a single depth. These issues can be addressed for clinical applications, especially if absolute in vivo dosimetry is desired, by making T1 mapping readily available on the Unity.

### Example 2-10

In vivo dosimetry has long been recognized as a valuable adjunct to radiation therapy. Unlike patient-specific QA procedures such as phantom measurements and redundant calculations, in vivo dosimeters can report the actual dose received by patients. The present study described a novel, MR-readable injectable hydrogel dosimeter having characteristics that enable real-time in vivo dosimetry for MRgRT. The injectable hydrogel dosimeter marries two valuable technologies in radiation therapy: an injectable, space-filling material that can increase the separation between a treatment target and an OAR and a real-time, in vivo, 3D dosimeter that can be interrogated externally and does not require removal to report the absorbed dose. This combination of desirable attributes leads to several opportunities, one of which is confirmation that an OAR is being adequately protected or possibly an early alert that an adjustment to the treatment plan is required. Unlike conventional in vivo dosimetry systems, the injectable dosimeter herein can report the dose from an intracavitary or interstitial location within or adjacent to the target volume. And unlike several electronic dosimeters, the system is biologically and MRI compatible and requires only a minimally invasive procedure for implantation. The injectable hydrogel dosimeter can also measure clinically relevant dose levels for multiple irradiations over several weeks. This means that the device can be interrogated during treatment delivery, immediately following a treatment fraction, or on a less-frequent basis, while providing valuable, spatially relevant dosimetric information.

### Enumerated Embodiments

In some aspects, the present invention is directed to the following non-limiting embodiments:

Embodiment 1: An injectable dosimeter composition comprising: a polymer gel; and a radiation dosimeter material distributed in at least a portion of the polymer gel, wherein the radiation dosimeter material generates a dosimetric signal in response to a radiation applied to the polymer gel, and wherein the injectable dosimeter composition is biocompatible.

Embodiment 2: The injectable dosimeter composition of Embodiment 1, wherein the polymer gel comprises a crosslinked polymer gel, optionally wherein the polymer gel is crosslinked via a small molecule crosslinker, a reactive functional group attached to the polymer chain, a photo cross-linking group attached to the polymer chain, or an enzyme-catalyzed cross-linking reaction.

Embodiment 3: The injectable dosimeter composition of any one of Embodiments 1-2, wherein the polymer gel comprises a hydrogel, optionally wherein the hydrogel comprises an alginate hydrogel, a carboxymethylcellulose hydrogel, a collagen hydrogel, a hyaluronic acid hydrogel, a polyethylene glycol (PEG) hydrogel, a poly (2-hydroxyethyl methacrylate) (pHEMA) hydrogel, a poly (2-hydroxypropyl methacrylate) hydrogel, a polymethylmethacrylate (PMMA) hydrogel, a poly-lactic acid (PLA) hydrogel, a polyacrylamide hydrogel, or combinations thereof.

Embodiment 4: The injectable dosimeter composition of any one of Embodiments 1-3, wherein the polymer gel comprises a crosslinked hyaluronic acid polymer.

Embodiment 5: The injectable dosimeter composition of any one of Embodiments 1-4, wherein the polymer gel comprises a hyaluronic acid polymer modified with a norbornene group and a hyaluronic acid polymer modified with a tetrazine group.

Embodiment 6: The injectable dosimeter composition of any one of Embodiments 1-5, wherein the polymer gel is partially polymerized.

Embodiment 7: The injectable dosimeter of any one of Embodiments 1-5, wherein the polymer gel is fully polymerized.

Embodiment 8: The injected dosimeter of Embodiment 7, which has been injected into the body of a subject.

Embodiment 9: The injectable dosimeter of any one of Embodiments 1-8, wherein the dosimetric signal generated in response to the radiation is substantially proportional to the radiation dose.

Embodiment 10: The injectable dosimeter of any one of Embodiments 1-9, wherein the dosimetric signal generated in response to the radiation is detectable by magnetic resonance imaging (MRI) or any optical imaging method.

Embodiment 11: The injectable dosimeter of any one of Embodiments 1-10, wherein the radiation comprises at least one selected from the group consisting of x-ray, gamma ray, electron beam, and proton beam.

Embodiment 12: The injectable dosimeter of any one of Embodiments 1-11, wherein the radiation dosimeter material comprises a transition metal element.

Embodiment 13: The injectable dosimeter of any one of Embodiments 1-12, wherein the radiation dosimeter material comprises Fe and/or Gd.

Embodiment 14: The injectable dosimeter of Embodiment 13, wherein the radiation dosimeter material comprises Fe (II), and wherein the radiation converts the Fe (II) to Fe (III).

Embodiment 15: The injectable dosimeter of any one of Embodiments 1-14, which is physically and chemically stable for about 1 day or more at a temperature of about 36° C. to about 38° C.

Embodiment 16: A method of evaluating radiotherapy administered to a subject, the method comprising: injecting the injectable dosimeter composition of any one of Embodiments 1-15 into the subject at a site near a site to be treated by the radiotherapy; administering the radiotherapy to the subject; and detecting the dosimetric signal in the radiation dosimeter material of the injectable dosimeter composition.

Embodiment 17: The method of Embodiment 15, wherein the injectable dosimeter composition is injected between the site to be treated by the radiotherapy and a site not to be treated by the radiotherapy.

Embodiment 18: The method of any one of Embodiments 16-17, further comprising, while administering the radiotherapy, adjusting the radiotherapy pattern and/or intensity based on the detected dosimetric signal.

Embodiment 19: The method of any one of Embodiments 16-18, further comprising establishing a relationship between a change in the dosimetry signal in the injectable dosimeter and a dose of radiation received by the injectable dosimeter.

Embodiment 20: A method of performing radiotherapy on a subject, the method comprising: injecting the injectable dosimeter composition of any one of Embodiments 1-15 into the subject at a site near a disease site of the subject; applying radiation to the disease site of the subject; and detecting the dosimetric signal in the radiation dosimeter material of the injectable dosimeter.

Embodiment 21: The method of Embodiment 20, wherein the injectable dosimeter composition is injected between the disease site and a site not to be treated by the radiotherapy.

Embodiment 22: The method of any one of Embodiments 20-21, further comprising, while applying the radiation, adjusting the radiation pattern and/or intensity based on the detected dosimetric signal.

Embodiment 23: The method of any one of Embodiments 20-22, further comprising establishing a relationship between a change in the dosimetry signal in the injectable dosimeter and a dose of radiation received by the injectable dosimeter.

Embodiment 24: A method of providing a spacer and/or a marker in a radiotherapy, the method comprising: injecting the injectable dosimeter of any one of Embodiments 1-15 near the site to be treated by the radiotherapy.

Embodiment 25: The method of Embodiment 24, wherein the injectable dosimeter composition is injected as a spacer between the site to be treated by the radiotherapy and a site not to be treated by the radiotherapy, thereby protecting the site not to be treated by the radiotherapy from radiation.

Embodiment 26: The method of Embodiment 24, wherein the injectable dosimeter composition is injected as a spacer to stabilize a site to be treated by the radiotherapy or a site not to be treated by the radiotherapy during the radiotherapy.

Embodiment 27: The method of Embodiment 24, wherein the injectable dosimeter is injected as a spacer that conforms to irregular surface of a site to be treated with radiotherapy.

Embodiment 28: The method of Embodiment 24, wherein the injectable dosimeter is injected as a marker to increase the contrast between a site to be treated by the radiotherapy and a site that is not to be treated by the radiotherapy.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An injectable dosimeter composition comprising:
a polymer gel; and
a radiation dosimeter material distributed in at least a portion of the polymer gel,
wherein the radiation dosimeter material generates a dosimetric signal in response to a radiation applied to the polymer gel, and
wherein the injectable dosimeter composition is biocompatible.

2. The injectable dosimeter composition of claim 1, wherein at least one of the following applies:
(a) the polymer gel comprises a crosslinked polymer gel, optionally wherein the polymer gel is crosslinked via a small molecule crosslinker, a reactive functional group attached to the polymer chain, a photo cross-linking group attached to the polymer chain, or an enzyme-catalyzed cross-linking reaction;
(b) the polymer gel comprises a hydrogel, optionally wherein the hydrogel comprises an alginate hydrogel, a carboxymethylcellulose hydrogel, a collagen hydrogel, a hyaluronic acid hydrogel, a polyethylene glycol (PEG) hydrogel, a poly (2-hydroxyethyl methacrylate) (pHEMA) hydrogel, a poly (2-hydroxypropyl methacrylate) hydrogel, a polymethylmethacrylate (PMMA) hydrogel, a poly-lactic acid (PLA) hydrogel, a polyacrylamide hydrogel, or combinations thereof;
(c) the polymer gel comprises a crosslinked hyaluronic acid polymer; or
(d) the polymer gel comprises a hyaluronic acid polymer modified with a norbornene group and a hyaluronic acid polymer modified with a tetrazine group.

3. (canceled)

4. (canceled)

5. (canceled)

6. The injectable dosimeter composition of claim 1, wherein the polymer gel is partially polymerized.

7. The injectable dosimeter of claim 1, wherein the polymer gel is fully polymerized.

8. The injected dosimeter of claim 7, which has been injected into the body of a subject.

9. The injectable dosimeter of claim 1, wherein at least one of the following applies:
(a) the dosimetric signal generated in response to the radiation is substantially proportional to the radiation dose;
(b) the dosimetric signal generated in response to the radiation is detectable by magnetic resonance imaging (MRI) or any optical imaging method;
(c) the radiation comprises at least one selected from the group consisting of x-ray, gamma ray, electron beam, and proton beam;
(e) the radiation dosimeter material comprises a transition metal element.

10. (canceled)

11. (canceled)

12. (canceled)

13. The injectable dosimeter of claim 1, wherein the radiation dosimeter material comprises Fe or Gd.

14. The injectable dosimeter of claim 13, wherein the radiation dosimeter material comprises Fe (II), and wherein the radiation converts the Fe (II) to Fe (III).

15. The injectable dosimeter of claim 1, which is physically and chemically stable for about 1 day or more at a temperature of about 36° C. to about 38° C.

16. A method of evaluating radiotherapy administered to a subject, the method comprising:
injecting the injectable dosimeter composition of claim 1 into the subject at a site near a site to be treated by the radiotherapy;
administering the radiotherapy to the subject; and
detecting the dosimetric signal in the radiation dosimeter material of the injectable dosimeter composition.

17. The method of claim 16, wherein the injectable dosimeter composition is injected between the site to be treated by the radiotherapy and a site not to be treated by the radiotherapy.

18. The method of claim 16, wherein at least one of the following applies:
(a) the method further comprises, while administering the radiotherapy, adjusting the radiotherapy pattern or intensity based on the detected dosimetric signal; or
(b) the method further comprises establishing a relationship between a change in the dosimetry signal in the injectable dosimeter and a dose of radiation received by the injectable dosimeter.

19. (canceled)

20. A method of performing radiotherapy on a subject, the method comprising:
injecting the injectable dosimeter composition of claim 1 into the subject at a site near a disease site of the subject;
applying radiation to the disease site of the subject; and
detecting the dosimetric signal in the radiation dosimeter material of the injectable dosimeter.

21. The method of claim 20, wherein at least one of the following applies:
(a) the injectable dosimeter composition is injected between the disease site and a site not to be treated by the radiotherapy;
(b) the method further comprises, while applying the radiation, adjusting the radiation pattern or intensity based on the detected dosimetric signal; or
(c) the method further comprises establishing a relationship between a change in the dosimetry signal in the injectable dosimeter and a dose of radiation received by the injectable dosimeter.

22. (canceled)

23. (canceled)

24. A method of providing a spacer or a marker in a radiotherapy, the method comprising:
injecting the injectable dosimeter of claim 1 near the site to be treated by the radiotherapy.

25. The method of claim 24, wherein the injectable dosimeter composition is injected as a spacer between the site to be treated by the radiotherapy and a site not to be treated by the radiotherapy, thereby protecting the site not to be treated by the radiotherapy from radiation.

26. The method of claim 24, wherein the injectable dosimeter composition is injected as a spacer to stabilize a site to be treated by the radiotherapy or a site not to be treated by the radiotherapy during the radiotherapy.

27. The method of claim 24, wherein the injectable dosimeter is injected as a spacer that conforms to irregular surface of a site to be treated with radiotherapy.

28. The method of claim 24, wherein the injectable dosimeter is injected as a marker to increase the contrast between a site to be treated by the radiotherapy and a site that is not to be treated by the radiotherapy.
